(19) United States
(12) Patent Application Publication
(10) Pub. No.: US 2009/0069557 A1
(43) Pub. Date: Mar. 12, 2009

(54) PREPARATION OF GEMCITABINE

(75) Inventors: Venkata Raghavendra Acharyulu Palle, Andhra Pradesh (IN); Sekhar Munaswamy Nariyam, Andhra Pradesh (IN); Veerender Murki, Andhra Pradesh (IN); Anirudha Arvind Waghmare, Maharashtra (IN); Vishnu Nandkishore Mundhada, Gujarat (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: Dr. Reddy's Laboratories Ltd., Hyderabad, AP (IN); Dr. Reddy's Laboratories, Inc., Hyderabad, AP (IN)

(21) Appl. No.: 12/161,844

(22) PCT Filed: Feb. 6, 2007
(86) PCT No.: PCT/US07/61687

§ 371 Date: Jul. 23, 2008

Related U.S. Application Data
(60) Provisional application No. 60/804,836, filed on Jun. 15, 2006.
(60) Provisional application No. 60/821,170, filed on Aug. 2, 2006.

(30) Foreign Application Priority Data
Feb. 6, 2006 (IN) 183/CHE/2006
May 18, 2006 (IN) 874/CHE/2006

(51) Int. Cl. C07H 19/06; C07D 307/04; C07H 1/00

(52) U.S. Cl. 536/28.51; 549/477; 549/476; 536/55.3

(57) ABSTRACT

A process for preparation of gemcitabine hydrochloride and purification thereof.

The present invention relates to a process for the preparation of gemcitabine and salts thereof. In an aspect, the invention relates to a process that provides gemcitabine hydrochloride free from its process-related impurities.

Gemcitabine hydrochloride is the adopted name for a drug compound chemically known as 2′-deoxy-2′,2′-difluorocytidine monohydrochloride (β-anomer) and having the structural Formula I.

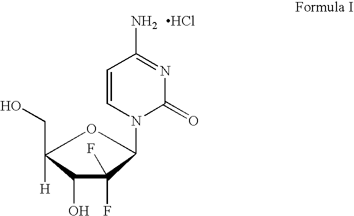

Gemcitabine hydrochloride is a nucleoside analogue that exhibits anti-tumor activity and is available in the market under the brand name GEMZAR® in the form of an injection. A vial of Gemzar contains gemcitabine hydrochloride equivalent to either 200 mg or 1 g of gemcitabine free base.

Methods for preparing gemcitabine are known in the art. For example U.S. Pat. No. 4,808,614 discloses gemcitabine hydrochloride, compositions containing gemcitabine hydrochloride and their use in the treatment of herpes viral infections. It also describes a process for the preparation of gemcitabine hydrochloride. The process discloses the use of hydrolysis reagents such as mildly acidic ion exchange resins for the conversion of compound of Formula III to compound of Formula IV of the present invention.

U.S. Pat. No. 5,223,608 discloses a process for the preparation of gemcitabine hydrochloride by using hydrolysis reagents such as strong acids in the preparation of 2-deoxy-2,2-difluoro-D-erythro-pentofuranos-1-ulose-3,5-dibenzoate.

International Application Publication No. WO 2005/095430 A1 discloses a process for the preparation of gemcitabine hydrochloride. This application describes the use of hydrolysis reagents like strong acids for the preparation of 2-deoxy-2,2-difluoro-D-erythro-pentofuranos-1-ulose-3,5-d ibenzoate and its purification process. This application also describes a process for the purification of gemcitabine hydrochloride by the dissolution of 95% enriched β-anomer of gemcitabine hydrochloride in water and isolated by using solvents like isopropyl alcohol or acetonitrile or acetone.

The above approaches employ mildly acidic ion exchange resins and strong acids as the hydrolyzing agents during the formation of unprotected or protected lactone. Both types of hydrolyzing agents suffer from the disadvantage of formation of a lactone ring, undesirable reaction products, impurities and often a lactone reverts back to its open chain precursor because of its sensitivity to strong acids and resins.

U.S. Pat. No. 5,945,547 discloses a process for the purification of gemcitabine hydrochloride with respect to its anomeric impurity, comprising dissolution of a 1:1 α/β anomeric mixture in hot water, followed by the addition of acetone at reflux, and cooling the solution to a temperature of about −10 to 50° C. The precipitated gemcitabine hydrochloride was collected and subjected to further purification by repeating the above process to afford purified β-anomeric gemcitabine hydrochloride.

International Application Publication No. WO 2006/095359 discloses a process for the preparation of gemcitabine hydrochloride. This application describes process for preparing gemcitabine by using a protecting group such as p-chloro or p-methyl or p-nitro benzoyl. This application also describes a process for the purification of gemcitabine hydrochloride, by slurrying 95% enriched β-anomer of gemcitabine hydrochloride in water and then the forming a solid with acetone. Gemcitabine hydrochloride was again purified with water and acetone or acetonitrile or isopropanol to obtain a 99.9% β-enriched gemcitabine hydrochloride.

International Application Publication No. 2006/092808 discloses a process for the purification of gemcitabine hydrochloride by using recrystallization with water and acetic acid to get 99.94% of the β-anomer from the original 95% of the β-anomer.

The present invention relates to a process for the preparation of intermediates, and improvements in the purification techniques to afford the desired β-anomer of gemcitabine hydrochloride substantially free from the α-anomer.

According to the present invention there is provided a convenient process for the preparation of gemcitabine hydrochloride and its intermediates with desired purity and yield by using better preparation techniques, which are simple, ecofriendly, cost-effective, robust and well suited for use on an industrial scale.

### SUMMARY OF THE INVENTION

The present invention provides a process for the preparation of gemcitabine hydrochloride and its intermediates.

In one aspect, the present invention provides a process for the preparation of gemcitabine or a salt thereof comprising:

i) conversion of 2-difluoro-3-hydroxy-3 (2,2-dimethyldioxalan-4-yl)propionate of Formula III to 2-desoxy-2,2-difluoro-1-oxoribose of Formula IV, using iodine and a lower alcoholic solvent, followed by azeotrophic distillation;

ii) protection of the hydroxy groups of the compound of Formula IV using tertiary-butyl di phenyl silyl chloride as a hydroxy-protecting reagent to afford 3,5-bis(tertiary-butyl di phenyl silyloxy)-2-deoxy-2,2-difluoro-1-oxoribose of Formula V;

iii) reduction of the compound Formula V using a suitable reducing reagent to afford 3,5-bis(tertiary-butyl di phenyl silyloxy)-2-deoxy-2,2-difluororibose of Formula VI;

iv) protection of the compound of Formula VI using an alkyl or aryl sulfonyl chloride, in the presence of a suitable base to afford 3,5-bis(tertiary-butyl di phenyl silyloxy)-1 methane sulfonyloxy-2-deoxy-2,2-difluororibose of Formula VIII;

v) condensation of the compound of Formula VII with the N-acetylcytosine compound of Formula VIII in the presence of a suitable base to afford 1-[2′-deoxy-2′,2′-difluoro-3′,5′-ribo furanose-3,5-bis(tertiary-butyl di phenyl silyloxy)cytocine of Formula IX; and

vi) deprotection of the compound of Formula IX using a suitable reagent to get gemcitabine, which can be subsequently converted into an acid addition salt by reacting with an acid.

In another aspect, the present invention relates to a process for the purification of gemcitabine hydrochloride to afford gemcitabine hydrochloride enriched with its β-anomer.

in an embodiment, a process for the purification of gemcitabine hydrochloride comprises:

a) providing a solution of gemcitabine hydrochloride in an aqueous solvent;

b) increasing the concentration of gemcitabine hydrochloride in the solution to cause precipitation;

c) isolating the compound enriched in the β-anomer.

An embodiment of the invention provides a process for preparing gemcitabine or a salt thereof, comprising reacting a compound having the Formula IV:

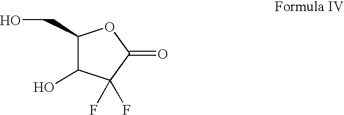

with t-butyldiphenylsilyl chloride, to form a compound having the Formula V.

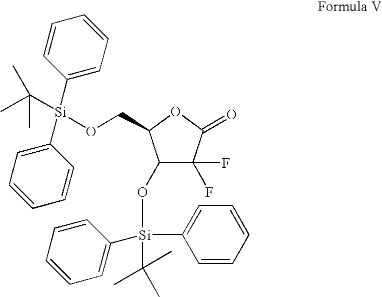

Another embodiment of the invention provides a compound having a formula:

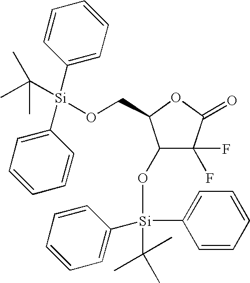

A further embodiment of the invention provides a compound having a formula:

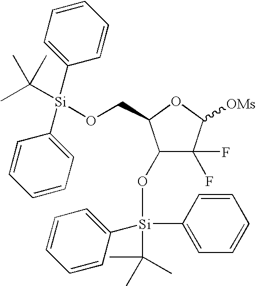

where “OMs” represents a methane sulfonyl group.

A further embodiment of the invention provides a compound having a formula:

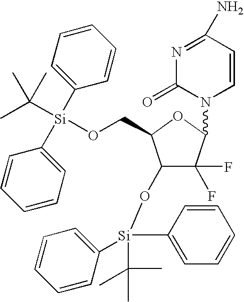

### BRIEF DESCRIPTION OF THE DRAWINGS

is an X-ray powder diffraction (XRPD) pattern of gemcitabine hydrochloride prepared according to Example 10.

FIG. 2 is a differential scanning calorimetry (DSC) curve of gemcitabine hydrochloride prepared according to Example 10.

FIG. 3 is an infrared absorption (IR) spectrum of gemcitabine hydrochloride prepared according to Example 10.

FIG. 4 is a thermogravimetric (TGA) curve of gemcitabine hydrochloride prepared according to Example 10.

FIG. 5 is an HPLC chromatogram of gemcitabine hydrochloride prepared according to Example 10.

FIG. 6 is a schematic representation of a process for preparing gemcitabine hydrochloride.

### DETAILED DESCRIPTION OF THE INVENTION

The present invention generally relates to a process for the preparation of gemcitabine hydrochloride and its intermediates.

In one aspect the present invention provides a process for the preparation of gemcitabine or a salt thereof comprising:

i) conversion of 2,2-difluoro-3-hydroxy-3 (2,2-dimethyldioxalan-4-yl)propionate of Formula III to 2-desoxy-2,2-difluoro-1-oxoribose of Formula IV, using iodine and lower alcoholic solvent, followed by azeotrophic distillation;

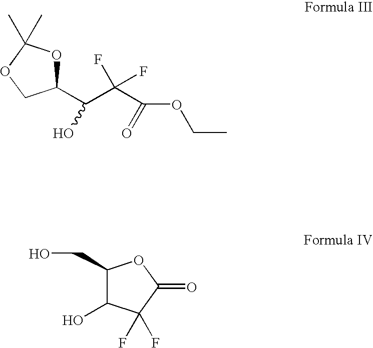

ii) protection of the hydroxy groups of the compound of Formula IV using tertiary-butyl di phenyl silyl chloride as a hydroxy-protecting reagent to afford 3,5-bis(tertiary-butyl di phenyl silyloxy)-2-deoxy-2,2-difluoro-1-oxoribose of Formula V;

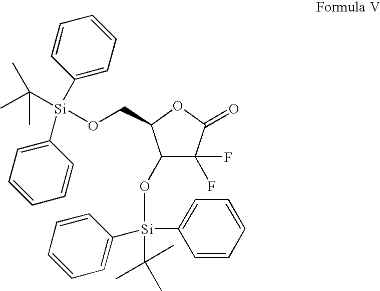

iii) reduction of the compound Formula V using a suitable reducing reagent to afford 3,5-bis(tertiary-butyl di phenyl silyloxy)-2-deoxy-2,2-difluororibose of Formula VI;

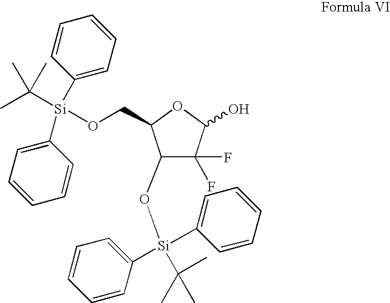

iv) protection of the compound of Formula VI using an alkyl or aryl sulfonyl chloride, in the presence of a suitable base to afford 3,5-bis(tertiary-butyl di phenyl silyloxy)-1 methane sulfonyloxy-2-deoxy-2,2-difluororibose of Formula VIII,

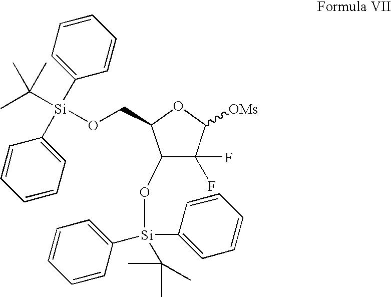

where “OMs” represents a methane sulfonyl group;

v) condensation of the compound of Formula VII with the N-acetylcytosine of Formula VIII in the presence of a suitable base to afford 1-[2′-deoxy-2′,2′-difluoro-3′,5′-ribo furanose-3,5-bis(tertiary-butyl di phenyl silyloxy) cytocine of Formula IX; and

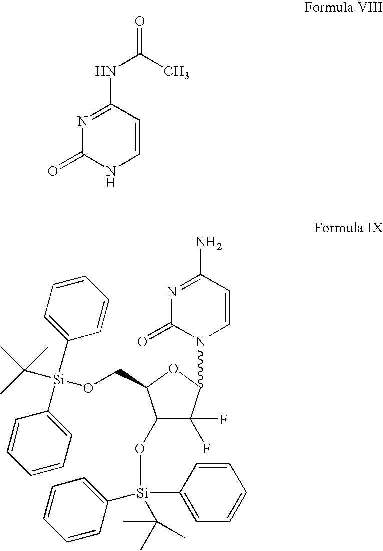

vi) deprotection of the compound of Formula IX using a suitable reagent to get gemcitabine, which can be subsequently converted into an acid addition salt by reacting with an acid.

Step i) involves conversion of 2,2-difluoro-3-hydroxy-3 (2,2-dimethyldioxalan-4-yl) propionate compound of Formula III to 2-desoxy-2,2-difluoro-1-oxoribose of Formula IV, using iodine and a lower alcohol solvent, followed by azeotrophic distillation.

Lower alcohol solvents that can be used in the process of step i) have 1 to about 6 carbon atoms, either straight chain or branced chain, and include methanol, ethanol, n-propanol, isopropanol, n-butanol and the like.

Suitable solvents which can be used for the reaction medium include lower alcohol (C₁-C₆) solvents, hydrocarbons such as toluene and the like.

Suitable temperatures for conducting the reaction range from about 20° C. to about 70° C.

After completion, the reaction mixture is quenched with water and sodium thiosulfate. A water immiscible solvent is added to the reaction mixture and subjected to azeotropic distillation to remove water and methanol. The distillation can be conducted at temperatures of about 50° C. to about 150° C.

Water immiscible solvents that can be used include hydrocarbons and halogenated hydrocarbons such as n-hexane, cyclohexane, n-heptane, toluene, chlorobenzene, 1,2-dichlorobenzene, xylenes and the like

The reaction mixture comprising the product may be used directly in the subsequent reaction step or suitably it can be concentrated to obtain a residue.

Step ii) involves protection of the hydroxy groups of the compound of Formula IV using tertiary-butyl di phenyl silyl chloride as a hydroxy-protecting reagent in the presence of a suitable base to afford 3,5-bis(tertiary-butyl di phenyl silyloxy)-2-deoxy-2,2-difluoro-1-oxoribose of Formula V.

Suitable alternative hydroxy protecting groups which can be also used in the above reaction include but are not limited to silyl hydroxy protecting groups such as tertiary-butyl diphenylsilyl, trimethylsilylchloride, isopropyldimethylsilyl, methyldiisopropylsilyl, triisopropylsilyl and the like, formyl, 2-chloroacetyl, benzyl, diphenylmethyl, triphenylmethyl, 4-nitrobenzyl, phenoxycarbonyl, t-butyl, methoxymethyl, phenyoxyacetyl, isobutyryl, ethoxycarbonyl, benzyloxycarbonyl and the like.

Suitable bases which can be used include but are not limited to organic bases such as pyridine, triethylamine, imidazole, 2,6-lutidine, 2,3-lutidine, 3,5-lutidine and the like.

Suitable solvents which can be used in the process of step ii) include but are not limited to: ethers such as tetrahydrofuran, 1,4-dioxane, diethyl ether, 1,2-dimethoxy ethane and the like; hydrocarbons such as n-hexane, cyclohexane, n-heptane, toluene, xylenes and the like; halogenated hydrocarbons such as chlorobenzene, 1,2-dichlorobenzene, and the like; aprotic polar solvents such as N,N-dimethyl formamide (DMF), dimethylsulfoxide, dimethylacetamide, acetonitrile and the like; ketones such as acetone, methyl isobutyl ketone, methyl tertiary butyl ketone, and the like; and mixtures thereof.

Suitable temperatures for conducting the reaction range from about 5° C. to about 50° C.

Step iii) involves reduction of the compound Formula V using a suitable reducing reagent to afford 3,5-bis(tertiary-butyl di phenyl silyloxy)-2-deoxy-2,2-difluororibose of Formula VI.

Suitable reducing reagents, which can be used, include but are not limited to sodium bis(2-methoxyethoxy) aluminum hydride (Vitride), sodiumborohydride (NaBH₄), lithium aluminium hydride (LiAlH₄), diisobutylaluminium hydride (DIBAL-H), and the like.

Suitable solvents which can be used include but are not limited to: ethers such as tetrahydrofuran, 1,4-dioxane, diethyl ether, 1,2-dimethoxy ethane and the like; hydrocarbons such as n-hexane, cyclohexane, n-heptane, toluene, xylenes and the like; halogenated hydrocarbons such as chlorobenzene, 1,2-dichlorobenzene, and the like; and mixtures thereof.

Suitable temperatures for conducting the reaction range from about −60° C. to about 100° C.

Step iv) involves protection of the compound of Formula VI using an alkyl or aryl sulfonyl chloride, in the presence of a suitable base to afford 3,5-bis (tertiary-butyl di phenyl silyloxy)-1 methane sulfonyloxy-2-deoxy-2,2-difluororibose of Formula VIII.

Suitable protecting groups which can be used for protecting the hydroxyl group include, but are not limited to alkyl and aryl sulfonyl chlorides such as methane sulfonyl chloride, benzene sulfonyl chloride and the like.

In an embodiment of the present invention the protecting group used for protecting the hydroxyl group of the compound of Formula VI is methanesulfonylchloride in the presence of base to afford the compound of Formula VIII.

Suitable bases, which can be used, include but are not limited to organic bases such as pyridine, triethylamine, diethyl amine, and the like.

Suitable solvents which can be used for the above reaction include but are not limited to: ethers such as tetrahydrofuran, 1,4-dioxane, diethyl ether, and 1,2-dimethoxy ethane and the like; hydrocarbons such as n-hexane, cyclohexane, n-heptane, toluene, xylenes, and the like; halogenated hydrocarbons such as chlorobenzene, 1,2-dichlorobenzene, and the like; aprotic polar solvents such as N,N-dimethyl formamide (DMF), dimethylsulfoxide, dimethylacetamide, acetonitrile and the like; esters such as ethyl acetate, isopropyl acetate and the like; ketones such as acetone, methyl isobutyl ketone, methyl tertiary-butyl ketone, and the like; and mixtures thereof.

Suitable temperatures for conducting the reaction range from about 0° C. to about 50° C.

Step v) involves condensation of the compound of Formula VII with the N-acetylcytosine compound of Formula VIII in the presence of a suitable base to afford 1-[2′-deoxy-2′,2′-difluoro-3′,5′-ribo furanose-3,5-bis(tertiary-butyl di phenyl silyloxy)cytocine of Formula IX.

Suitable solvents which can be used in the above reaction include but are not limited to: ethers such as tetrahydrofuran, 1,4-dioxane, diethyl ether, 1,2-dimethoxy ethane and the like; hydrocarbons such as n-hexane, cyclohexane, n-heptane, toluene, xylenes and the like; halogenated hydrocarbons such as chlorobenzene, 1,2-dichlorobenzene, and the like; esters such as ethyl acetate, isopropyl acetate and the like; ketones such as acetone, methyl isobutyl ketone, methyl tertiary-butyl ketone, and the like; and mixtures thereof.

Suitable bases, which can be used, include, but are not limited to organic bases such as pyridine, triethylamine, hexamethyldisilazane, trimethylsilyltriflate, and the like, and combinations thereof.

Suitable temperatures for conducting the reaction range from about 20° C. to about 120° C.

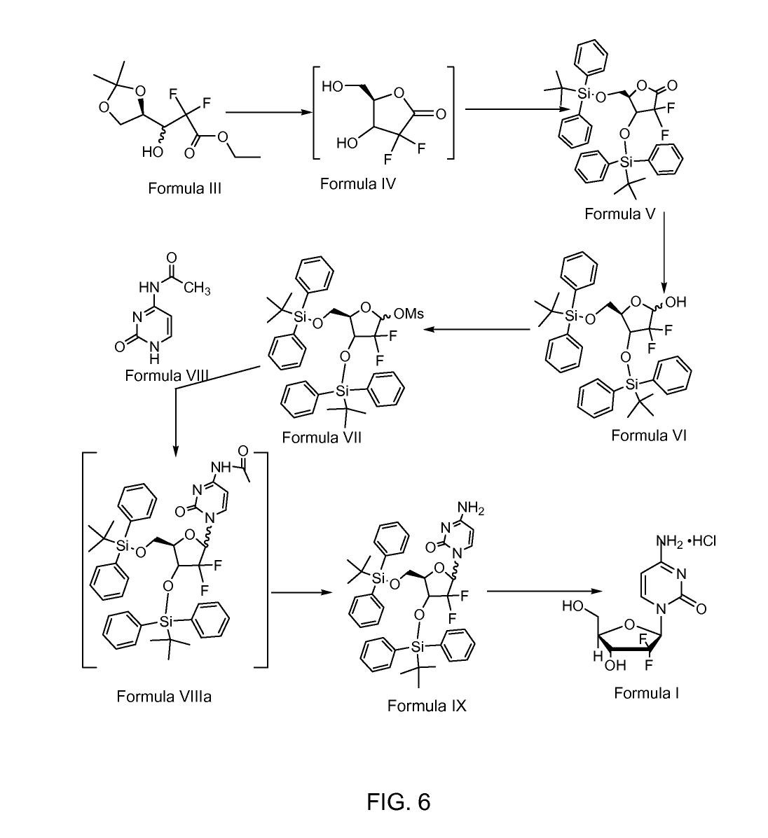

Optionally, 1-[2′-deoxy-2′,2′-difluoro-3′,5′-ribo furanose-3,5-bis(tertiary-butyl di phenyl silyloxy) N-acetyl cytocine (Formula VIIIa In FIG. 6) that forms as an intermediate is not isolated. When this option is used, the reaction mixture containing this compound is progressed directly to the next step.

Step vi) involves deprotection of the compound of Formula IX using a suitable reagent to get gemcitabine, which can be subsequently converted into an acid addition salt by reacting with an acid.

Suitable deprotecting reagents which can be used include ammonium fluoride, tert-butyl ammonium fluoride, ammonia, acetyl chloride, dilute hydrochloric acid and the like.

The solvents used in the above reactions include but are not limited to: water; alcohols such as methanol, ethanol, ethanol hydrochloride, n-propanol, isopropanol, isopropyl alcohol, n-butanol and the like; halogenated solvents such as dichloromethane, dichloroethane, chloroform, chlorobenzene, 1,2-dichlorobenzene and the like; ethers such as tetrahydrofuran, 1,4-dioxane, diethyl ether, 1,2-dimethoxy ethane and the like; hydrocarbons such as n-hexane, cyclohexane, n-heptane, toluene, xylenes and the like; aprotic polar solvents such as N,N-dimethylformamide (DMF), dimethylsulfoxide, dimethylacetamide, acetonitrile and the like; esters such as ethyl acetate, isopropyl acetate and the like; ketones such as acetone, methyl isobutyl ketone, methyl tertiary-butyl ketone, and the like; and mixtures thereof.

Suitable temperatures for conducting the reaction range from about −10° C. to about 70° C.

Gemcitabine base obtained can be converted into a desired pharmaceutically acceptable acid addition salt by reacting with a suitable acid.

Suitable pharmaceutically acceptable acids which can be used include, but are not limited to: inorganic acids such as hydrochloric acid, hydrobromic acid, and hydroiodic acid; and organic acids such as acetic acid, tartaric acid, oxalic acid, and the like.

Optionally, one or more of sequential steps i) to iv) are carried out without isolating intermediate compounds. In one embodiment of the invention, step i) is carried out without isolating the intermediate, followed by isolation of the compound of Formula V.

Optionally, the wet cake obtained at various stages of the process can be further dried. Drying can be suitably carried out in a tray dryer, vacuum oven, air oven, fluidized bed drier, spin flash dryer, flash dryer and the like. The drying can be carried out at temperatures of about 35° C. to about 70° C. The drying can be carried out for any desired time periods to achieve the desired purity such as from about 1 to 20 hours, or longer.

The whole process is represented schematically in FIG. 6.

In another aspect, the present invention relates to a process for the purification of gemcitabine hydrochloride to afford gemcitabine hydrochloride enriched with its β-anomer.

An embodiment of the process for the purification of gemcitabine hydrochloride comprises:

a) providing a solution of gemcitabine hydrochloride in an aqueous solvent;

b) increasing the concentration of gemcitabine hydrochloride in the solution to cause precipitation; and

c) isolating the compound enriched in the β-anomer.

Step a) involves providing a solution of gemcitabine hydrochloride in an aqueous solvent.

The solution of gemcitabine hydrochloride is obtained by dissolving it in water or it can be obtained from a previous processing step where gemcitabine hydrochloride is formed. Any form of gemcitabine hydrochloride is acceptable for forming the solution, such as any crystalline or amorphous form of gemcitabine hydrochloride.

Gemcitabine hydrochloride for the purpose of dissolution can be prepared through methods known in the art or by the above-mentioned processes.

The concentration of anomeric salt mixture in the solution is not critical as long as sufficient water is employed to ensure total dissolution. The amount of water employed is usually kept small so as to avoid excessive product loss during crystallization and isolation. The quantity of water used for the isolation of the beta anomer is frequently about 1 to about 12 times to the weight of gemcitabine hydrochloride.

The solution can be prepared at a temperatures ranging from about 0° C. to about 100° C. Depending on the quantity of solvent taken, it may dissolve at 25 to 100° C., or the solution may need to be heated to elevated temperatures of about 50° C. to 100° C.

The solution can be optionally treated with activated charcoal to enhance the color of the compound, followed by filtration through a medium such as through a flux calcined diatomaceous earth (Hyflow) bed to remove the carbon.

The preferred quantity of charcoal carbon used in the isolation of the beta anomer with improved colour is about 0.1 to about 10 times the weight of anomeric α/β mixture.

The carbon treatment can be conducted either at the dissolution temperatures or after cooling the solution to lower temperatures.

Step b) involves increasing the concentration of gemcitabine hydrochloride in the solution to cause precipitation.

Concentration may be carried out suitably using evaporation, atmospheric distillation or distillation under vacuum.

Distillation of the solvent may be conducted under a vacuum of about 100 mm Hg to about 720 mm Hg at temperatures of about 40° C. to about 70° C. Any temperature and vacuum conditions can be used as long as concentration occurs without increase in the impurity levels.

Concentration of the solution can be carried out to an extent where the precipitation of the gemcitabine hydrochloride begins from the solution, converting the solution into slurry. Generally, concentration will be terminated when the ratio of solvent to gemcitabine hydrochloride becomes about 1:1 to about 1:5.

The reaction mixture may be maintained further at temperatures lower than the concentration temperatures such as, for example, below about 40° C. to about 45° C., for a period of time as required for a more complete isolation of the product. The exact cooling temperature and time required for complete crystallization can be readily determined by a person skilled in the art and will also depend on parameters such as concentration and temperature of the solution or slurry.

Step c) involves isolating the said compound enriched in said β-anomer.

The solid isolation can be conducted by techniques such as filtering, decanting, centrifuging and the like, or by filtering under an inert atmosphere using gases such as for example nitrogen and the like.

The wet cake obtained in step c) may optionally be further dried. Drying can be suitably carried out in a tray dryer, vacuum oven, air oven, fluidized bed drier, spin flash dryer, flash dryer and the like. The drying can be carried out at temperatures of about 35° C. to about 70° C. The drying can be carried out for any time periods necessary for obtaining a desired purity, such as from about 1 to 20 hours, or longer.

In a particular embodiment of the invention the above described process of the invention can be adapted to form the basis of a continuous crystallization process. The purity of the product obtained in step c) is checked to see the percentage of alpha-anomer impurity. If the impurity has not been reduced to the desired levels such as below 0.1%, as determined by high performance liquid chromatography (HPLC), then the steps a) to c) are repeated with the wet material obtained in step c). When the desired purity is attained at step c), the cycle is stopped.

Thus there is established a cycle of operations, which can be repeated indefinitely thereby adapting the process of the invention to a continuous process with obvious attendant advantages on the commercial scale.

The purified gemcitabine hydrochloride obtained above contains less than 0.1%, or less than 0.01%, of either of the cytosine impurity or the α-anomer.

Gemcitabine hydrochloride obtained by the above process was analyzed using HPLC according to the process described in United States Pharmacopeia 29; NF 24, 2005 (USP), pages 990-991, as described in Table 1.

TABLE 1
HPLC method

| Column and | Zorbax Rx C8 250 × 4.6 mm, 5μ | | | |
|---|---|---|---|---|
| Packing: | | | | |
| Buffer: | 13.8 g NaH₂PO₄•H₂O and 2.5 ml of phosphoric acid | | | |
| | was taken in 1000 ml of milli-Q water and filtered | | | |
| | through 0.45μ membrane filter. Solution pH was | | | |
| | between 2.4-2.6. | | | |
| Mobile Phase | Buffer used as mobile phase. | | | |
| A: | | | | |
| Mobile Phase | Filtered and degassed methanol. | | | |
| B: | | | | |
| Gradient: | Time (in | Solution A | Solution B | |
| | minutes) | (% v/v) | (% v/v) | Elution |
| | 0-8 | 97 | 3 | Isocratic |
| | 8-13 | 97-50 | 3-50 | Linear |
| | | | | gradient |
| | 13-20 | 50 | 50 | Isocratic |
| | 20-25 | 50-97 | 50-3 | Re- |
| | | | | equilibration |
| | 25-30 | 97 | 3 | Equilibration |

| | |
|---|---|
| Flow rate: | 1.2 ml/min |
| Wavelength of | 275 nm by UV |
| detection: | |
| Temperature: | 25 ± 2° C. |
| Injection | 20 μL |
| volume: | |
| Diluent: | Milli-Q water |
| Run time: | 30 minutes |

| IMPURITY NAME | RRT |
|---|---|
| α-Anomer impurity | 0.67 |
| Cytosine impurity | 0.37 |

The relative retention time (“RRT”) values for impurities are referenced to the retention time of gemcitabine, which is assigned a value of 1.

Crystalline gemcitabine hydrochloride obtained by the process of present invention is characterized by its X-ray powder diffraction (“XRPD”) pattern, differential scanning calorimetry (“DSC”) curve, and/or infrared (“IR”) absorption spectrum.

Crystalline gemcitabine hydrochloride obtained in the present invention is characterized by its XRPD pattern. All XRPD data reported herein were obtained using Cu Kα radiation, having the wavelength 1.541 Å and were obtained using a Bruker Axe D8 Advance Powder X-ray Diffractometer.

Crystalline gemcitabine hydrochloride is characterized by an XRPD diffraction pattern substantially in accordance with FIG. 1, having characteristic peaks at about 9.6, 11.4, 13.7, 19.1, 22.9, 24.0, 26.6, and 30.7, ±0.2 degrees 2 theta.

Differential scanning calorimetric analysis was carried out in a DSC Q1000 model from TA Instruments with a ramp of 5° C./minute with a modulation time of 60 seconds and a modulation temperature of ±1° C. The starting temperature was 0° C. and ending temperature was 200° C.

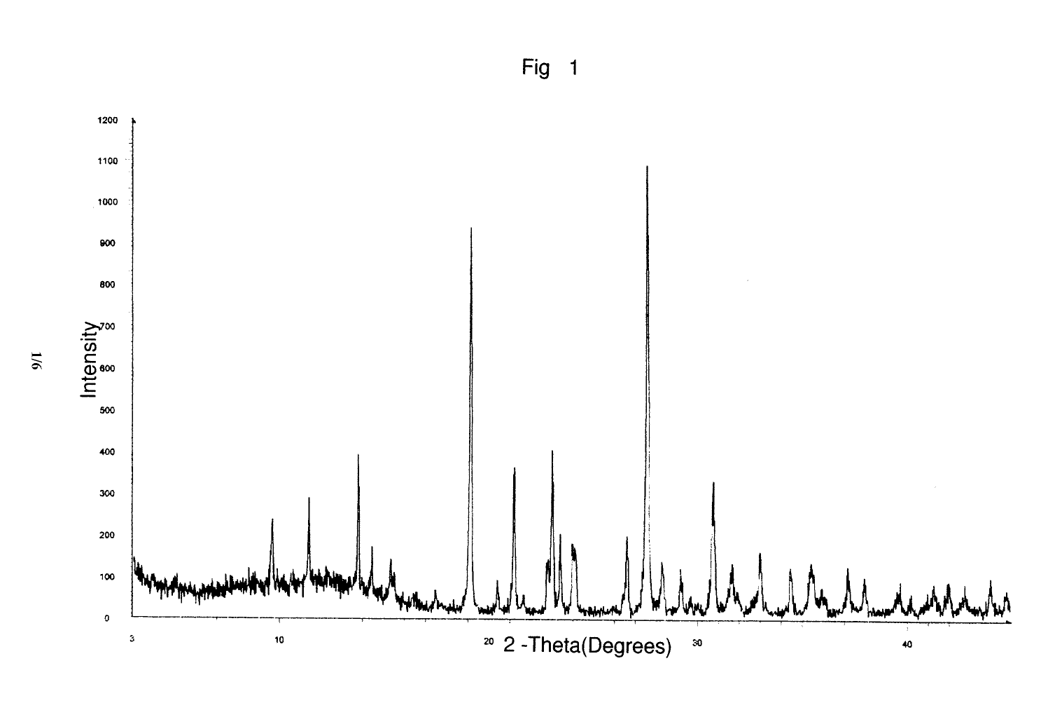

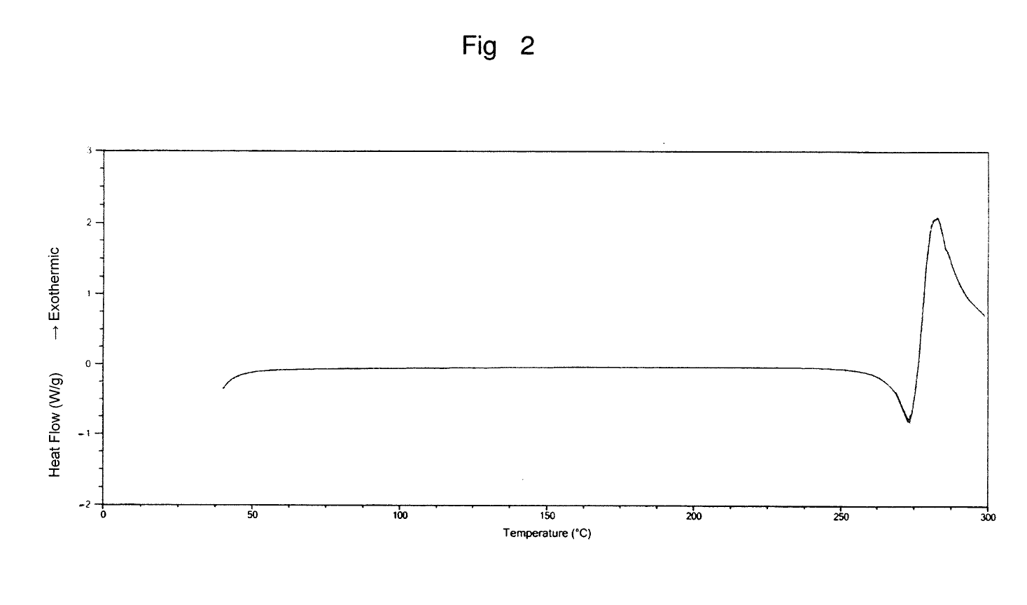

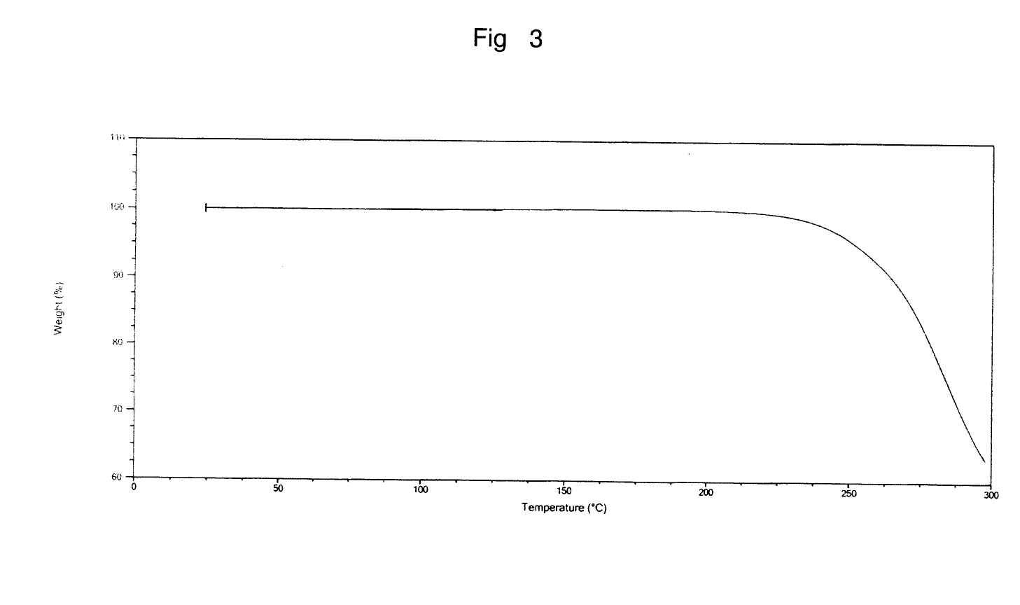

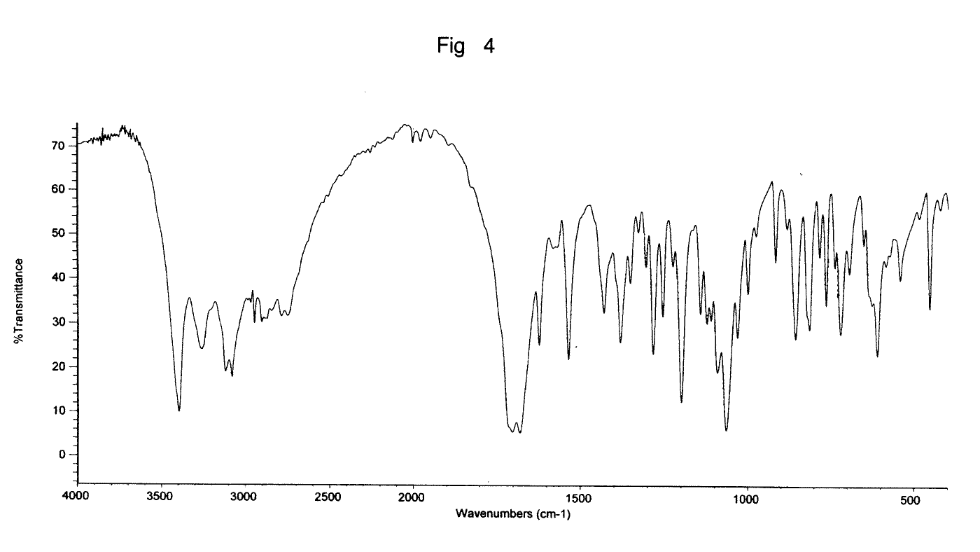

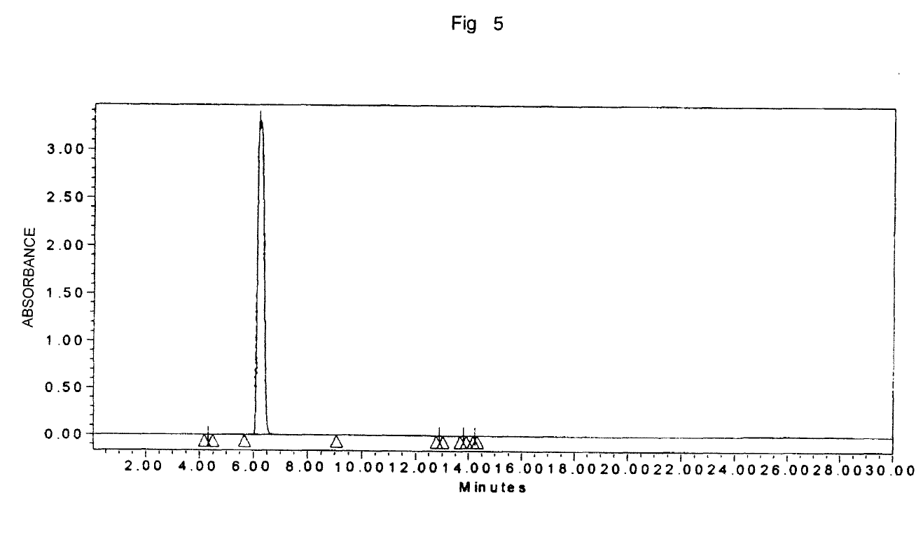

Crystalline gemcitabine hydrochloride has a characteristic differential scanning calorimetry curve substantially in accordance with FIG. 2, having an endothermic peak at 259-274° C.

The infrared absorption (IR) spectrum of gemcitabine hydrochloride has been recorded on a Perkin Elmer System Spectrum 1 model spectrophotometer, between 450 cm⁻¹ and 4000 cm⁻¹, with a resolution of 4 cm⁻¹ in a potassium bromide pellet, the test compound being at the concentration of 1% by mass.

Crystalline gemcitabine hydrochloride is characterized by an IR spectrum substantially in accordance with FIG. 2. Crystalline gemcitabine hydrochloride is characterized by an IR spectrum having characteristic peaks at about 3392, 3259, 3117, 3078, 1679, 1535, 1283, 1199, 1065, 856, and 814, ±5 cm⁻¹.

In yet another aspect the invention provides gemcitabine hydrochloride substantially free of residual solvents.

Gemcitabine hydrochloride obtained using the process of the present invention has amount of residual solvent content that is within the limits given by the International Conference on Harmonization of Technical Requirements for Registration of Pharmaceuticals for Human Use (“ICH”) guidelines. The guideline solvent level depends on the type of solvent but is not more than about 5000 ppm, or about 4000 ppm, or about 3000 ppm.

Gemcitabine hydrochloride obtained in this invention contains: less than about 100 ppm, or less than about 500 ppm, of acetone; less than about 100 ppm, or less than about 500 ppm, of isopropanol; and less than about 100 ppm, or less than about 500 ppm, of dichloromethane.

The dried product can optionally be milled to get the required particle size. Milling or micronization can be performed prior to drying, or after the completion of drying of the product. The milling operation reduces the size of particles and increases surface area of particles by colliding particles with each other at high velocities.

In an embodiment, gemcitabine hydrochloride obtained by the process of the present invention has a particle size distribution of: D₉₀ less than about 600 μm, or about 400 μm, or about 300 μm; D₅₀ less than about 400 μm, or about 300 μm, or about 300 μm; and D₁₀ less than 200 μm, or about 100 μm, or about 50 μm.

The D₁₀, D₅₀ and D₉₀ values are useful ways for indicating a particle size distribution. D₉₀ refers to the value for the particle size for which at least 90 volume percent of the particles have a size smaller than the value given. Likewise D₅₀ and D₁₀ refer to the values for the particle size for which 50 volume percent, and 10 volume percent, respectively, of the particles have a size smaller than the value given. Methods for determining D₁₀, D₅₀ and D₉₀ include laser light diffraction, such as using equipment from Malvern Instruments Ltd. (Malvern, Worcestershire, United Kingdom). There is no specific lower limit for any of the D values.

Certain specific aspects and embodiments of the present invention will be explained in more detail with reference to the following examples, which are provided by way of illustration only and should not be construed as limiting the scope of the invention in any manner.

### EXAMPLE 1

### PREPARATION OF 3,5-BIS(TERTIARY-BUTYL DI PHENYL SILYLOXY)-2-DEOXY-2,2-DIFLUORO-1-OXORIBOSE (FORMULA V)

100 g of ethyl 2,2-difluoro-3-hydroxy-3 (2,2-dimethyldioxalan-4-yl)propionate of Formula III, 1000 ml of methanol and 10 g of iodine were charged into a clean and dry round bottom flask followed by stirring overnight at 27° C. A 10% aqueous solution of sodium thiosulphate (22.5 g of sodium thiosulphate dissolved in 225 ml of water) was added to the above reaction mixture at about 27° C. over about 30 minutes followed by distillation of solvent from the reaction mixture at about 80° C. Subsequently 500 ml of toluene was charged into the obtained reaction mixture and then distilled off under atmospheric pressure at 95° C. The above step was repeated 5 times until the boiling point temperature of the reaction mixture was 110° C. Then the reaction mixture was distilled under a vacuum of 650 mm Hg at 50° C. to afford the 2-desoxy-2,2-difluoro-1-oxoribose compound of Formula IV as a residue.

The residue obtained from the above reaction was dissolved in 1320 ml of N,N-dimethyl formamide (DMF) under a nitrogen atmosphere at about 27° C. 80.3 g of imidazole and 300 ml of tertiary-butyldiphenylsilyl chloride (TBDPSi-Cl) were charged into the above resultant reaction mixture at 27° C., and stirred overnight at 27° C. The reaction completion was checked using thin layer chromatography (TLC) and the reaction mixture cooled to 15° C. 2600 ml of water was added at 27° C. with simultaneous stirring followed by extraction with 2×1000 ml of petroleum ether. Organic and aqueous layers were separated followed by washing the organic layer with 3×1000 ml of water and then the organic layer was distilled completely at 48° C. under vacuum. The obtained residue was purified by column chromatography by using petroleum ether and ethyl acetate solvent systems to afford 212.7 g of title compound having 93.49% purity by HPLC.

¹H NMR (400 MHz, DMSO-d₆, δ in ppm): 0.8 (9H), 1.1 (9H), 4.5 (2H), 3.4 (1H), 3.7 (9H).

MASS: m/z 662.4 (M+NH₄).

### EXAMPLE 2

### PREPARATION OF 3,5-BIS(TERTIARY-BUTYL DI PHENYL SILYLOXY)-2-DEOXY-2,2-DIFLUORORIBOSE (FORMULA VI)

243 g of 3,5-bis(tertiary-butyl di phenyl silyloxy)-2-deoxy-2,2-difluoro-1-oxoribose of Formula V and 2400 ml of tetrahydrofuran (THF) were charged into a round bottom flask under a nitrogen atmosphere at about 27° C. with simultaneous stirring. The reaction mixture was cooled to −45° C. and 192 ml of sodium bis(2-methoxyethoxy) aluminum hydride (Vitride) was added to above reaction mixture. The obtained reaction mixture was stirred for about 1 hour, 45 minutes and checked by thin layer chromatography to confirm the completion of the reaction. After completion of the reaction, 486 ml of saturated ammonium chloride solution was charged into above resultant reaction solution at about −45° C. and the temperature raised to 27° C. The obtained suspension was filtered and the aqueous layer from the filtrate was extracted with 1215 ml of ethyl acetate and 486 ml of brine solution followed by the separation of organic and aqueous layers. 1215 ml of ethyl acetate was charged to the aqueous layer and organic and aqueous layers were separated. Finally the total organic layer was distilled completely under vacuum to afford 245 g of title compound.

¹H NMR (400 MHz, DMSO-d₆, δ in ppm): 0.9 (9H), 1.1 (9H), 5.1 (1H)

MASS: m/z 664.4 (M+NH₄)

### EXAMPLE 3

### PREPARATION OF 3,5-BIS(TERTIARY-BUTYL Di PHENYL SILYLOXY)-1 METHANE SULFONYLOXY-2-DEOXY-2,2-DIFLUORORIBOSE (Formula VII)

245 g of 3,5-bis(tertiary-butyl di phenyl silyloxy)-2-deoxy-2,2-difluororibose of Formula VI and 2450 ml of dichloromethane were charged into a round bottom flask under a nitrogen atmosphere with simultaneous stirring. The reaction mixture was cooled to 0° C. and 55.5 ml of triethylamine and 18.6 ml of methane sulfonyl chloride were charged to the above reaction mixture. The reaction mixture temperature was allowed to rise to 27° C. and was stirred for about 2.5 hours. Reaction completion was checked using thin layer chromatography. 1225 ml of water was charged into above resultant reaction mixture and the two layers were separated. The resultant organic layer was distilled completely at 45° C. under vacuum to get a gummy solid. Petroleum ether was charged to above obtained gummy solid and cooled to 2° C. The obtained suspension was stirred for 3 hours and filtered the solid followed by suction drying to afford 191 g of the title compound.

¹H NMR (400 MHz, DMSO-d₆, δ in ppm): 5.9 (1H), 0.8 (9H), 1.1 (9H), 3.1 (3H).

MASS: m/z 742.2 (M+NH₄).

### EXAMPLE 4

### PREPARATION OF 1-[2′-DEOXY-2′,2′-DIFLUORO-3′,5′-RIBO FURANOSE-3,5-BIS(TERTIARY-BUTYL Di PHENYL SILYLOXY)CYTOCINE (FORMULA IX)

89 g of N-acetyl cytosine was charged into a round bottom flask under a nitrogen atmosphere. 525 ml of hexamethyl disilazane (HMDS) was added into the above flask at 27° C. 18.5 ml of trimethyl silyl chloride was added to the above reaction mixture and 132 ml of trimethylsilyltriflate (TMS triflate) was added. 105 g of the mesylated compound of Formula VII was charged to above reaction mixture and the reaction mixture heated to 93° C. and stirred for 3 hours at 93° C. The reaction mixture was cooled to about 35° C. and 500 ml of dichloromethane was charged. The obtained reaction solution was added to cooled water at 9° C. over the period of 40 minutes. Filtered the obtained suspension and washed the wet cake with 210 ml of dichloromethane. Separated organic and aqueous layers from the obtained filtrate and extracted the aqueous layer with 500 ml of dichloromethane. Finally combined the organic layers and washed the organic layer with 2×500 ml of water. The total organic layer was distilled completely at 44° C. under a vacuum of 600 mm Hg to afford 105 g of title compound.

### EXAMPLE 5

### Preparation of Gemcitabine

105 g of 1-[2′-deoxy-2′,2′-difluoro-3′,5′-ribo furanose-3,5-bis(tertiary-butyl di phenyl silyloxy)-cytocine of Formula IX, which was prepared in Example 4, was dissolved in 1500 ml of methanol. 72 g of ammonium fluoride was added to the reaction solution and then heated to 65° C. (reflux). The reaction mixture was maintained for about 3 hours at reflux to complete the reaction and then the solvent was distilled at 48° C. under a vacuum of 600 mm Hg. 500 ml of isopropyl alcohol was charged to the residue and cooled to 5° C., and then maintained for 10 minutes at 5° C. The obtained reaction mixture was filtered and the solid washed with 200 ml of isopropyl alcohol. The obtained filtrate was distilled completely and then 500 ml of water and 500 ml of dichloromethane were charged to the residue, and the organic and aqueous layers were separated. The obtained aqueous layer was washed with 2×500 ml of dichloromethane. 4 g of charcoal was added to obtained aqueous solution and stirred for 20 minutes, and then filtered through a Hyflow bed and the bed washed with 50 ml of water. The obtained filtrate was concentrated completely under vacuum at 47° C. to afford 27 g of title compound having purity 96.16% (α: 62.05%, β: 34.11%).

### EXAMPLE 6

### Preparation of Gemcitabine Hydrochloride of Formula I

75 ml of isopropyl alcohol and 25 g of gemcitabine base obtained from Example 5 were charged into a round bottom flask and then the isopropyl alcohol solvent was distilled off under a vacuum of 600 mm Hg at 47° C. The above step was repeated two more times. Again 75 ml of isopropyl alcohol was charged to above obtained residue and heated to 60° C. 10 ml of 36% aqueous hydrochloric acid was added to the above reaction solution and then allowed to cool to 27° C. Again cooled the reaction suspension to −4° C. and stirred for 6.5 hours at −4° C. The obtained solid suspension was filtered and the wet solid washed with 75 ml of acetone. The solid was dried at 39° C. under vacuum for 3 hours to afford 9.4 g of title compound.

Purity by HPLC: 98.5% (α: 11.19%, β: 87.31%).

### EXAMPLE 7

### Recrystallization of Gemcitabine Hydrochloride

0.5 g of gemcitabine hydrochloride and 1.5 ml of water were charged into round bottom flask and stirred the reaction mixture for 6 hours at 27° C. Then the reaction suspension was filtered and washed the solid with 2 ml of acetone, and then dried for 3 hours at 40° C. to afford 0.37 g of title compound.

Purity by HPLC: 99.81% of β-anomer.

% of α anomer: 0.05%.

### EXAMPLE 8

### Purification of Gemcitabine Hydrochloride

26.4 L (3 volumes) of demineralized water was taken into a reactor, and heated to a temperature of about 82° C. 6.6 Kg gemcitabine hydrochloride crude anomeric salt mixture having a ratio of α:β about 1:1 by HPLC was added to the above reactor with simultaneous stirring till the formation of clear solution. The reaction solution was cooled to about 23° C., followed by filtration of separated solid under a nitrogen atmosphere and the solid was washed with 5 L of acetone. The resultant solid was suction dried for about 1 hour under nitrogen pressure to afford 2.7 Kg of gemcitabine hydrochloride.

Purity by HPLC: 98.1%; α-anomer: 1.7%.

### EXAMPLE 9

### Purification of Gemcitabine Hydrochloride

860 ml (10 volumes) of purified water was charged into a reactor, along with 86 g of gemcitabine hydrochloride crude anomeric mixture having a ratio of α:β about 1:1. The mixture was heated to a temperature of about 38° C. with simultaneous stirring to form a clear solution. 0.1 volume of charcoal carbon was added to the reaction mixture at 38° C. with stirring for about 30 minutes. The reaction mixture was filtered through Hyflow followed by washing with 3×30 ml of demineralized water at room temperature. The obtained filtrate was concentrated to 3 volumes of the reaction mixture at 46° C. under a vacuum of 600 mm Hg and then cooled to 17° C. with simultaneous stirring for about 30 minutes at 18° C. The reaction mixture was filtered followed by washing with 50 ml of acetone at 16° C. and suction drying for 30 minutes. The solid obtained was dried at a temperature of about 40° C. for about 4 hours under vacuum 600 mm Hg to afford 31 g of gemcitabine hydrochloride.

Purity by HPLC: 95.6%; α-anomer: 6.8%.

### EXAMPLE 10

### Purification of Gemcitabine Hydrochloride

342 ml (12 volumes) of purified water was charged into a reactor, along with 28.5 g of gemcitabine hydrochloride having a purity 95.6%, and heated to a temperature about 37° C. with simultaneous stirring to form a clear solution. 0.1 volume of charcoal was added to the reaction mixture at 37° C. with stirring for about 30 minutes. The reaction mixture was filtered through a Hyflow bed followed by washing with 3×25 ml of demineralized water at room temperature. The obtained filtrate was concentrated to 2 volumes of the reaction mixture at 47° C. under a vacuum of 600 mm Hg and then cooled to 30° C. The reaction suspension was filtered followed by washing with 31 ml of acetone at 27° C. The above process was repeated one time and finally the solid obtained was dried at a temperature of 40° C. for about 4 hours under a vacuum of 600 mm Hg to afford 24 g of gemcitabine hydrochloride.

Purity by HPLC: 99.96%; α-anomer: 0.01%.

Optical rotation: [α]_{D}²⁰ (10 mg/ml aqueous solution): +47.1°.

1. A process for preparing gemcitabine or a salt thereof, comprising reacting a compound having Formula IV: 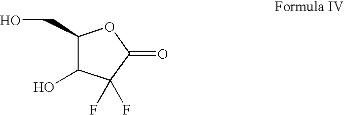
with t-butyldiphenylsilyl chloride, to form a compound having Formula V:

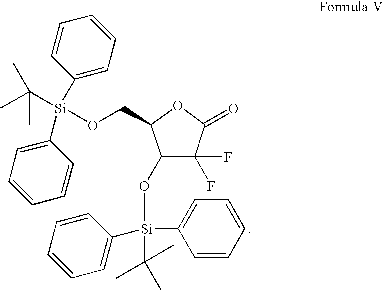

2. The process of claim 1, wherein a compound having Formula IV is prepared by reacting a compound having Formula III: 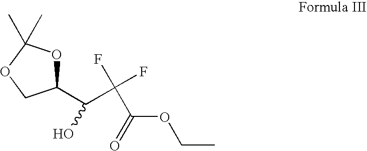
with iodine, in an alcohol solvent.

3. The process of claim 1, further comprising reacting a compound having Formula V with a reducing reagent to form a compound having Formula VI:

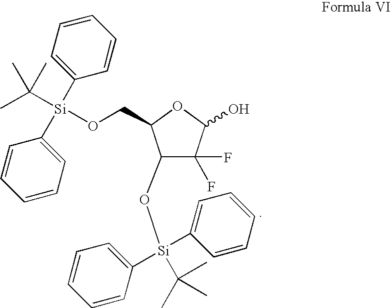

4. The process of claim 3, further comprising reacting the compound having Formula VI with methane sulfonyl chloride to form a compound having Formula VII: 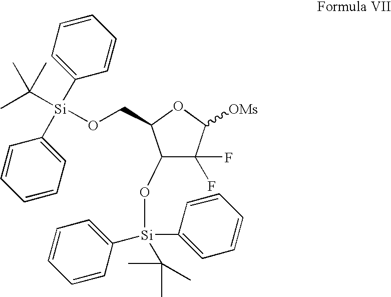
where “OMs” represents a methane sulfonyl group.

5. The process of claim 4, further comprising condensation of the compound having the Formula VII with N-acetylcytosine to form a compound having Formula IX:

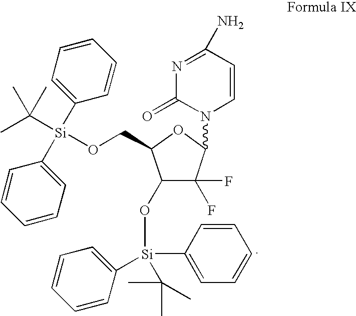

6. The process of claim 5, wherein a compound having Formula VIIIa: 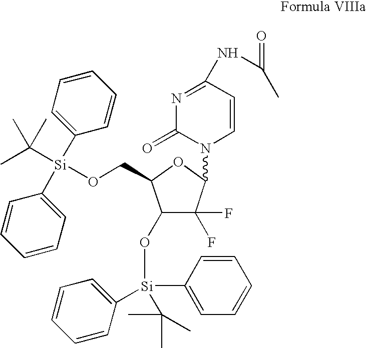
is formed during condensation, but is not isolated.

7. The process of claim 5, further comprising deprotecting a compound having Formula IX to form gemcitabine.

8. A compound having a formula:

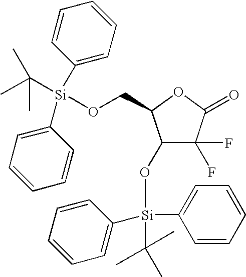

9. A compound having a formula: 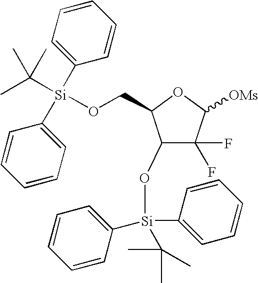
where “OMs” represents a methane sulfonyl group.

10. A compound having a formula:

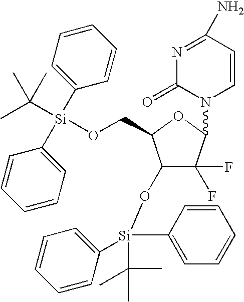

11. A process for enrichment of a β-anomer of gemcitabine hydrochloride, comprising:
a) providing a solution of gemcitabine hydrochloride in an aqueous solvent;
b) increasing the concentration of solute in the solution to cause precipitation;
c) isolating gemcitabine hydrochloride enriched in said β-anomer.

12. The process of claim 11, wherein the weight ratio of water to gemcitabine hydrochloride in the solution of a) is about 3:1 to about 12:1.

13. The process of claim 11, wherein the solution of a) is treated with activated charcoal.

14. The process of claim 11, wherein increasing concentration comprises removing a portion of water.

15. The process of claim 11, wherein concentration increasing is continued until a weight ratio of water to gemcitabine hydrochloride is less than about 3:1.

16. The process of claim 11, wherein isolation of said β-anomer enriched gemcitabine hydrochloride is carried out at temperatures about 0° C. to about 10° C.

17. The process of claim 11, wherein β-anomer enriched gemcitabine hydrochloride contains not less than about 99.7% of said β-anomer.

18. The process of claim 11, wherein β-anomer enriched gemcitabine hydrochloride contains not less than about 99.9% of said β-anomer.
